(19) United States
(12) Patent Application Publication
(10) Pub. No.: US 2012/0220991 A1
(43) Pub. Date: Aug. 30, 2012

(54) AQUEOUS COMPOSITION COMPRISING GOLD NANOPARTICLES, SERUM ALBUMIN AND/OR COLLAGEN FOR LASER TISSUE WELDING

(75) Inventors: Philippe Jenny, Pessac (FR); Marc Bernard, Leognan (FR)

(73) Assignees: Marc BERNARD, Leognan (FR); CREASPINE, Pessac (FR); Franck TRICOT, Talence (FR)

(21) Appl. No.: 13/508,215

(22) PCT Filed: Aug. 27, 2010
(86) PCT No.: PCT/FR2010/051792

§ 371 Date: May 4, 2012

(30) Foreign Application Priority Data
Nov. 9, 2009 (FR) 09/05378

(51) Int. Cl. A61B 18/20; C09J 189/00; B82Y 5/00

(52) U.S. Cl. 606/3; 106/157.2; 977/773; 977/908

(57) ABSTRACT

The invention relates to an aqueous composition used in the laser repair treatment of fibrous connective tissue, including: gold nanoparticles in a concentration of between 1.5×10⁹ particles per ml and 1×10¹⁰ particles per ml; and albumin and/or collagen in a concentration of between 30 wt % and 45 wt %. The invention also relates to an assembly including such a composition.

### TECHNICAL FIELD

The present invention concerns an aqueous composition used in the repair treatment of fibrous connective tissue.

A further subject of the invention is an assembly used for the repair treatment of fibrous connective tissue.

### BRIEF DISCUSSION OF RELATED ART

Fibrous connective tissue is relatively solid supporting tissue whose role is to protect the body organs it surrounds. Fibrous connective tissue can be predominantly made up of collagen, such as cartilage, dermis, ligaments, tendons and cornea.

Over time, the fibrous connective tissues may weaken to the point of fissuring or tearing thereby generating tissue defects in their structure through which the organ structure present inside the connective tissue may migrate, or resulting in abnormal, painful mobility of an element or fragment.

In cases of disc herniation for example, the central portion of the spinal disc (called the <<nucleus pulposis>>) escapes out of the surrounding ligamentous ring (“annulus fibrosis”) via a fissure in the said ring.

Usually, the orifice that is the cause of this hernia is not repaired during surgery which treats the protrusion of disc material and pain.

It is known for example to use invasive surgical techniques consisting of suturing the tissue to be repaired. Said techniques are traumatising for the tissues surrounding the tissue to be repaired and only allow imperfect closing of the orifice since they are made in damaged tissue that is mechanically weakened.

There are other more specific methods for repairing the said tissue defects using the insertion of polymer staples on the lesion, a mesh or a metal barrier affixed to the injured part. These added elements require a relatively invasive technique and generate the risk that the mechanical repair will not be durable in damaged tissue.

However, all these techniques close the fissure or tear superficially and do not allow the prevention or only imperfectly prevent painful post-operative recovery and recurrent migration through the repaired tissues.

In the laboratory it is known to use biological or cyanoacrylate glue either alone or in combination with a standard suture. However, this technique called the <<Gold standard>> cannot be used by a surgeon since the glue is not biocompatible.

A repair technique is known that is used for some types of tissues such as the viscera and eyes, which is based on the use of laser. The laser can be adjusted so that it locally heats the target tissue in precise manner. The heating generated is such that it destroys the tissue at the impact surface. It is therefore possible to break up a tumour or to make a very precise incision. Laser can also be used with lesser energy, capable of generating heating that is not so strong and is localised so as to obtain bonding or welding of biological tissues.

Insofar as laser heats locally and precisely and at a chosen depth, the tissues through which it passes are less traumatised than when using a conventional surgical technique requiring the incision of part of the surrounding tissues. In addition, a laser beam can be conveyed via optical fibres allowing micro-invasive techniques even percutaneous techniques that are distinctly less traumatising than standard surgical techniques.

For the repair and welding of fibrous connective tissue, it is necessary to combine the laser with a biocompatible composition to repair the said tissues similar to cementing. This composition must also allow a sealed repair to be obtained which is durable to avoid repeat surgery.

For the treatment of fibrous connective tissues by laser, there is therefore a need for a composition which allows the closing of tissue defects, such as fissures or tears, whilst allowing the stabilisation thereof to prevent the re-opening of these defects.

### BRIEF SUMMARY

The invention provides a composition which can be used for the repair of fibrous connective tissue and allows efficient and stable repair of the tissue defects without the aforementioned disadvantages.

For this purpose, according to a first aspect, the subject of the invention is an aqueous composition for use in the repair treatment of fibrous connective tissue using laser, comprising:
gold nanoparticles at a concentration of more than 1.5·10⁹ particles per ml and strictly less than 1·10¹⁰ particles per ml, and
albumin and/or collagen at concentration of between 30% and 45% by weight.

The composition of the invention is particularly suitable for the repair of fibrous connective tissue using laser. The concentration of the gold nanoparticles and of the albumin and/or collagen allows the generation by these two components of a more fluid-tight seal that is more durable than in the prior art. Surprisingly, outside these concentration ranges the weld is either not obtained or has a poor seal that is scarcely durable.

Within these concentration ranges, the cross-linking of albumin and/or collagen effectively occurs within an exposure time that is compatible with surgical use, namely between 30 seconds and 40 seconds. This cross-linking time allows the ensured sealing of the weld.

In addition, if the concentration of gold nanoparticles is lowered to below 1.5·10⁹ particles per ml, the necessary exposure time to obtain a weld is increased and hence less sealing is achieved.

On the other hand, at a concentration of more than 1·10¹⁰ particles per ml of gold nanoparticles, the heating related to welding becomes increasingly more difficult to control. In this case, the heat becomes increasingly stronger having the effect of reducing the adhesion between the added albumin and/or collagen and the fibrous connective tissue, in particular the annulus fibrosis of a spinal disc which deteriorates under the effect of heat. On this account, the seal of the weld is lessened. If exposure is continued, the induced heat destroys the cells of the fibrous connective tissue but also the surrounding cells.

If the weight concentration of the albumin and/or collagen is lowered to below 30%, the cross-linking thereof no longer forms a sufficiently resistant film to ensure the fluid-tightness of the weld.

If the weight concentration of the albumin and/or collagen is increased to above 45%, cross-linking is too strong and no longer occurs with the fibrous connective tissue, thereby reducing fluid-tightness.

The gold nanoparticles of the composition of the invention are adapted to absorb the laser wavelength by restoring energy in the form of heat which generates the cross-linking of the albumin and/or collagen of the composition. On cross-linking, the albumin and/or the collagen form a biocompatible matrix, filling the tissue defect such as a fissure or tear, and adhere to the walls of the fissure of the connective tissue. Therefore, the composition of the invention used at the concentrations such as indicated allows the maintaining of sufficient gold nanoparticles to allow the desired absorption of the laser wavelength in order to heat sufficiently the albumin and/or collagen for cross-linking thereof, these being in sufficient quantity to obtain a durable sealed weld. The particular composition of the invention allows the tissue defects to be stabilised at depth and does not only superficially close these defects as is the case in the prior art.

Therefore the closing and stabilisation are performed concomitantly in a single step and in precise manner.

In addition, the gold nanoparticles are chemically inert and do not produce any sub-product that is potentially toxic for the surrounding tissues. The albumin and collagen advantageously allow a cement matrix to be formed which maintains its strength over time, and they do not in turn produce a toxic sub-product at the time of their cross-linking.

According to other characteristics of the invention, the composition of the invention comprises one or more of the following optional characteristics taken alone or in any possible combination:
the albumin and/or collagen are of bovine or human origin, to ensure good biocompatibility with the tissue to be repaired;
the gold nanoparticles are of spherical, triangular or rod shape so that they can guarantee the desired absorption having regard to <<red shift>>;
the largest dimension of the gold nanoparticles is between 3 nm and 250 nm, which allows reduced heat diffusion and concentrates the heat at the weld interface;
the fibrous connective tissue is a cartilage, a ligament or a tendon.

According to another aspect, the subject of the invention is an assembly used comprising:
an aqueous composition according to the invention;
a laser emitting at a wavelength of between 0.78 μm and 1.4 μm.

The assembly of the invention is particularly suitable for efficient, stable repair of fibrous connective tissue. The laser used emits a light beam at a wavelength which is capable of passing through the surrounding tissues without damaging them whilst reaching the desired depth. With the composition of the invention it is possible to lower the wavelength by means of the presence of the chromophore gold nanoparticles. In addition, the composition is biocompatible with the surrounding tissues even once this composition has been denatured by contact with the laser. On this account, the repair and consolidation obtained with the assembly of the invention are concentrated on the damaged tissues and do not touch the surrounding tissues.

According to other characteristics of the invention, the assembly of the invention comprises one or more of the following optional characteristics taken along or in any possible combination:
the laser emits at a wavelength of about 808 nm which prevents damage to surrounding tissues;
the laser is pulsed or continuous;
the laser is configured to have a beam of diameter between 3 mm² and 7 mm² which allows precise repair of small tissue surfaces;
the laser is associated with a collimator and a fibre optical system allowing for easy management of the light beam;
the laser is configured to have an angle of incidence to the normal of the impact surface on the fibrous tissue of between 45° and 60° which provides for an optimal angle of incidence in the composition of the invention.

### BRIEF DESCRIPTION OF THE DRAWINGS

The invention will be better understood on reading the following non-limiting description given with reference to the appended Figures;

FIG. 1 is a schematic cross-section of healthy spinal disc;

FIG. 2 is a schematic cross-section of a spinal disc that is herniated;

FIG. 3 is a schematic perspective view of the assembly of the invention used to repair an incision made in a spinal disc;

FIG. 4 is a schematic cross-section of a spinal disc with an open incision;

FIG. 5 is a schematic cross-section of a spinal disc having an incision repaired with the laser technique using the assembly of the invention;

FIG. 6 is a schematic cross-section of a spinal disc having an incision repaired with the laser technique using the assembly of the invention, comprising an annular passageway in which a tube is inserted connected to a pressurized syringe allowing the injection of a saline solution containing ethylene blue into the nucleus.

### DETAILED DESCRIPTION

The present invention allows the repair of tissue defects of the fibrous connective tissue such as cartilage e.g. the medial or lateral meniscus of the knee, ligaments such as the annulus fibrosis of the spinal disc, the dermis, tendons, cornea.

Tissue defects are typically in the forms of tears or fissures which induce in-depth deterioration of the tissue.

The aqueous composition of the invention is therefore used for the repair testament of the fibrous connective tissue using laser and contains:
gold nanoparticles at a concentration of between 1.5·10⁹ particles per ml and 1·10¹⁰ particles per ml, and
albumin and/or collagen at a concentration of between 30 weight % and 45 weight %.

The composition of the invention is particularly suitable for the repair of fibrous connective tissue using laser. A fibrous connective tissue may effectively have tissue defects such as tears or fissures.

The gold nanoparticles of the composition of the invention are particularly adapted to absorb the wavelength of the laser and to restore the energy in the form of heat which generates the cross-linking of the albumin and/or collagen of the composition of the invention. On cross-linking, the albumin and/or collagen form a biocompatible matrix which fills the tissue defect such as a fissure or tear. Therefore, the composition of the invention used with the laser allows stabilising of the tissue defects at depth and does not only superficially close these defects as is the case in the prior art.

Therefore in a single step it is possible to stabilise the tissue defect in durable manner with a composition comprising non-toxic constituents and which does not produce any harmful sub-products for the surrounding tissues.

The concentration of the gold nanoparticles and of the albumin and/or collagen allows these two components to generate a more fluid-tight and more durable weld than in the prior art. Surprisingly, outside the concentration ranges, the weld is either not obtained or it is not or only scarcely sealed and durable.

Within these concentration ranges, the cross-linking of the albumin and/or collagen takes place within an exposure time that is compatible with surgical use, namely between 30 seconds and 40 seconds. This cross-linking time allows the ensured sealing of the weld.

Also, if the concentration of the gold nanoparticles is lowered to below 1.5·10⁹ particles per ml, the exposure time required to obtain a weld is increased, and less sealing is achieved.

On the other hand, at a concentration of more than 1·10¹⁰ particles per ml of gold nanoparticles, the heating related to welding becomes increasingly more difficult to control. In this case the heat becomes increasingly stronger, the effect of which is to reduce the adhesion between the added albumin and collagen and the fibrous connective tissue, in particular the annulus fibrosis of a spinal disc which deteriorates under the effect of heat. On this account, the fluid-tightness of the weld is reduced. If exposure is continued, the induced heat destroys the cells of the fibrous connective tissue but also the surrounding cells.

If the weight concentration of the albumin and/or collagen is lowered to below 30%, the cross-linking thereof no longer forms a sufficiently resistant film to ensure the seal of the weld.

If the weight concentration of the albumin and/or collagen is increased to above 45%, the cross-linking is too strong and no longer takes place with the fibrous connective tissue, which reduces the fluid-tightness.

The albumin and/or collagen have the advantage of improving the strength of the weld formed in the tissue defect and of obtaining a reliable weld. After cross-linking, the albumin and collagen act like glue which forms a binding matrix among the collagen fibres already present in the fibrous connective tissue.

In addition, by using collagen, the structural support is improved whilst increasing the rate of post-weld repair since collagen is part of the endogenous materials of connective tissues.

The albumin and/or collagen can be of bovine or human origin which allows the ensured good biocompatibility with the tissue to be repaired.

The gold nanoparticles allow the power of the laser to be reduced by selecting a short wavelength, generally shorter than 820 nm. The gold nanoparticles are capable of selectively absorbing wavelengths of the light spectrum for conversion to heat and transfer of the heat to the surrounding collagen and/or albumin. The gold nanoparticles allow the wavelength of the laser beam to be reduced so as to limit the interaction between the light beam and the tissues. This is particularly important for a heterogeneous medium in which tissues surrounding the tissue to be repaired are sensitive to wavelengths shorter than the wavelength tolerated by the tissue to be repaired. For example, the case may be mentioned of the repair of the annulus fibrosis of a spinal disc which has herniated.

In addition, gold nanoparticles have the advantage of being chemically inert and non-toxic. Also, they do not produce any degradation sub-products that are harmful or toxic for tissue cells.

By <<nanoparticles>> is meant nanoparticles whose largest dimension is between 3 nm and 1000 nm, even between 3 nm and 250 nm. The small size of these nanoparticles allows a reduction in heat diffusion which occurs from the treatment site, and concentrates heat at the weld interface thereby minimising the damage caused to the tissues surrounding the tissue to be repaired.

The gold nanoparticles are typically gold nanoparticles formed by agglomerating gold atoms.

The gold nanoparticles may be hollow or they may be used to coat a silica or silicate core.

Gold nanoparticles can be functionalised by coating with polyethylene glycol (PEG). In this manner, advantageously, the nanoparticles are stabilised in the aqueous composition preventing flocculation of these nanoparticles which provides for easy management of the nanoparticles.

The gold nanoparticles may be of any suitable shape to guarantee the desired absorption whilst limiting <<red shift>> corresponding to spectral absorption shift towards the red wavelengths, namely less than 800 nm. Therefore these particles may be of spherical, triangular or rod shape.

The composition of the invention is prepared by diluting the gold nanoparticles in deionised water to obtain a concentration of between 1.5·10⁹ particles per ml and 1·10¹⁰ particles per mml, and albumin and/or collagen, so as to obtain a weight concentration relative to the final composition of between 30% and 4 5%.

The composition of the invention may be more or less viscous and in particular it may be in gel form. For a gel, it is advantageously possible to inject the composition of the invention into the desired point and the said composition remains at the injected point without flowing or leaking which allows repair at greater depth of the tissue compared with a liquid composition. The gel advantageously allows the treatment of the tissue defect in vivo preventing body fluid from carrying away the composition before the weld is made. In this manner surgical procedure is simplified, avoiding having to maintain <<dry>> the region to be treated.

According to another aspect, the invention also concerns an assembly used in the repair treatment of fibrous connective tissue, comprising:
the aqueous composition of the invention; and
a laser emitting at a wavelength in the near infrared, namely at between 0.78 μm and 1.4 μm.

The assembly of the invention is particularly suitable for efficient stable repair of fibrous connective tissue. The laser and the composition used for the invention are advantageously innocuous for the surrounding tissues. The laser beam passes through the different tissues before reaching the tissue to be repaired without causing detrimental interaction with these different tissues such as heating. In addition the composition is adapted to be biocompatible and not to generate any toxic sub-products. The repair and consolidation achieved with the assembly of the invention are therefore concentrated on the damaged tissue.

More specifically, the laser is focused on the composition of the invention at the point where the repair is to take place, namely in the tissue defect. The gold nanoparticles absorb the wavelength emitted by the laser beam and emit heat captured by the molecules of albumin and/or collagen surrounding the gold nanoparticles. Under the effect of the heat, the macromolecules of albumin and/or collagen are polymerised and become a glue matrix which hardens filling the fissures or tears in the fibrous connective tissue. Therefore in the repaired part of the fibrous connective tissue, durable structural cohesion of the said tissue is ensured which fills the tissue defect.

The laser emits at a wavelength of about 808 nm i.e. an optimal wavelength which the gold nanoparticles are capable of absorbing to return the heat to the albumin and/or collagen. In addition, at this wavelength there is weaker interaction with the tissues surrounding the tissue to be repaired.

The laser may be a diode laser for example.

The laser may be pulsed or continuous. A pulsed laser, when applicable, allows the sending of a light pulse having greater energy power, and concentrates the energy over a very short time whilst not inducing a rise in temperature higher than a continuous laser.

The power of the laser is between 10 kW and 12 Kw.

The laser may be configured so that it has a beam of diameter between 3 mm² and 7 mm², or a diameter adapted to the size of the region to be treated whilst maintaining an identical surface power (per cm²) which allows precise repair of small tissue surfaces.

The laser can be associated with a collimator which allows focusing on a given point of the surface.

During a surgical operation, the surgeon is not always able to bring the beam to the point of the lesion but must position the laser beam towards the lesion by passing through body organs or bones such as the spine for example to treat disc herniation. For such purpose, it is possible to cause the light beam to pass through an endoscope or a hand-piece close to the region to be repaired.

The laser can also be associated with a fibre optical system allowing easier management of the beam and the adjustment of the light spot derived from the light beam onto the point of the tissue which is to be repaired;

The laser can be configured to have an angle of incidence to the normal of the impact surface of the fibrous tissue of between 45° and 90°, preferably between 45° and 60°, which allows optimal incidence to be obtained through the composition of the invention.

According to one variant it is possible to glue a patch containing collagen and/or albumin directly onto the tear or fissure. The use of a said patch allows a further increase in the mechanical resistance of the weld of up to 50% and prevents loss of the aqueous composition if it is a hydrated composition.

### Comparative Example of the Repair Strength of a Lesion of Disc Herniation Type.

Description of Disc Herniation

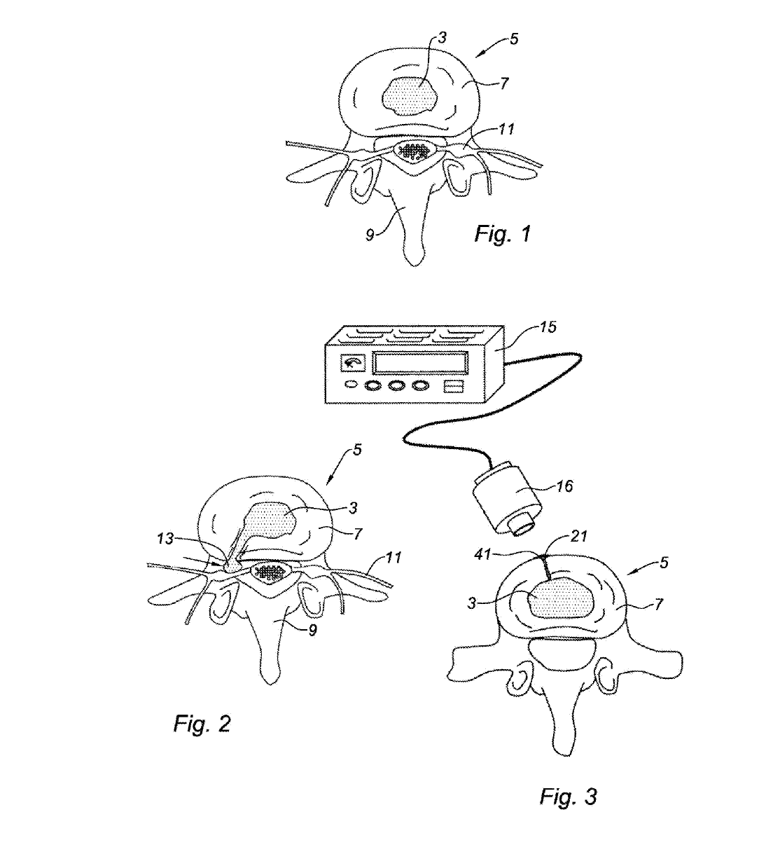

As illustrated in FIG. 1, a healthy or normal spinal disc 5 comprises a centre 3 (<<nucleus pulposis>>) surrounded by a spinal ligament 7 (<<annulus fibrosis>>). The spinal disc is also surrounded by vertebrae 9 which house the spinal cord from which the nerve roots 11 emerge. The annulus fibrosis 7 is surrounded by muscle and tissue. As illustrated in FIG. 2, a disc herniation corresponds to a protrusion 13 of the centre 3 through the spinal ligament 7.

Preparation of an Example of an Aqueous Composition of the Invention

The composition used in this example is prepared as follows.

The albumin solution is prepared by diluting about 2 g of bovine albumin serum in about 5 ml of deionised water. The final concentration obtained is about 40% by weight, namely 40 g per 100 ml.

The solution of gold nanoparticles is prepared by centrifuging at 10 000 rpm for 30 minutes a solution composed of about 11 ml of deionised water with a concentration of about 2.47·10⁹ particles. A deposit of gold nanoparticles is obtained on which the deionised water is a supernatant. About 10.61 ml of supernatant water is removed and the deposit is dissolved in the remaining water. An aqueous solution is then obtained containing a concentration of gold nanoparticles of about 7·10¹⁰ particles per ml.

About 142.9 μl of the solution of gold nanoparticles is taken and placed in a 1.5 ml tube whose volume is completed with about 857.1 μl of final albumin solution to obtain a final solution of about 1 ml having a nanoparticle concentration of about 1·10¹⁰ nanoparticles per ml and an albumin weight concentration of about 34%.

Laser Used

A medical diode laser is used emitting at about 808 nm and with a power of about 7 kW connected to a diode laser source 15, a laser driver and a temperature controller. In the embodiment shown in FIG. 3, the diode laser, the diode laser source, the laser driver and the temperature controller are contained in one same receptacle 15. The laser is coupled with a collimator 16 forming a light spot having a diameter of about 3.1 mm, and fibre optical cabling 17.

The light output point of the light beam can be fixed to a metal rod arranged at an angle of about 50° to the perpendicular of the impact surface of the annulus fibrosis to be repaired, the said surface being arranged at about 10 cm from the output of the light beam from the optical fibres.

Two thermocouples are connected to a temperature data recorder. The thermocouple of about 0.4 mm in diameter can be inserted into the annulus to a depth of about 55 mm starting from the region to be repaired and on the two sides of the tissue defect. The temperature is recorded in real time and reaches a maximum value of about 45° C.

Preparation of Swine Spinal Discs

The spinal discs are taken from pigs weighing from 40 kg to 80 kg, within 4 hours after their death.

The sampled discs are cleaned of all meat using a scalpel in particular.

The spinal discs are frozen to about −18° C. for storage until use.

Preparation of the Samples

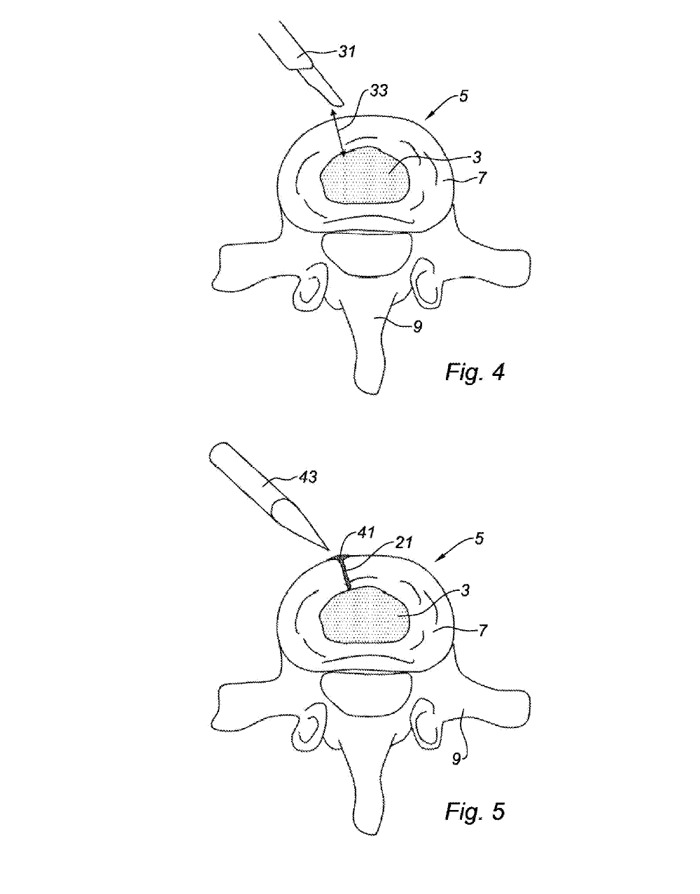

The repair of a disc herniation is simulated on swine spinal discs making an incision in the annulus fibrosis 6 (see FIG. 4). The incision is either non-repaired or repaired 21 (see FIG. 5) following two different methods.

As illustrated in FIG. 4, the incision is made using a scalpel 31 radial fashion (arrow 33) relative to the centre 3.

A first reference disc is prepared with an open incision, namely not repaired.

A second disc is prepared by repairing the incision with the <<Gold standard>> technique, namely the direct injection into the incision of two drops of cyanoacrylate glue injected into the incision, the incision having been closed by suture points.

As illustrated in FIG. 5, a third disc 5 is prepared by repairing the incision 21 with the assembly of the invention in the following manner. After making the incision, the composition 41 of the invention is injected directly into the said incision 21 using suitable means 43. The laser is then used as indicated above, focusing the beam on the incision to achieve the weld.

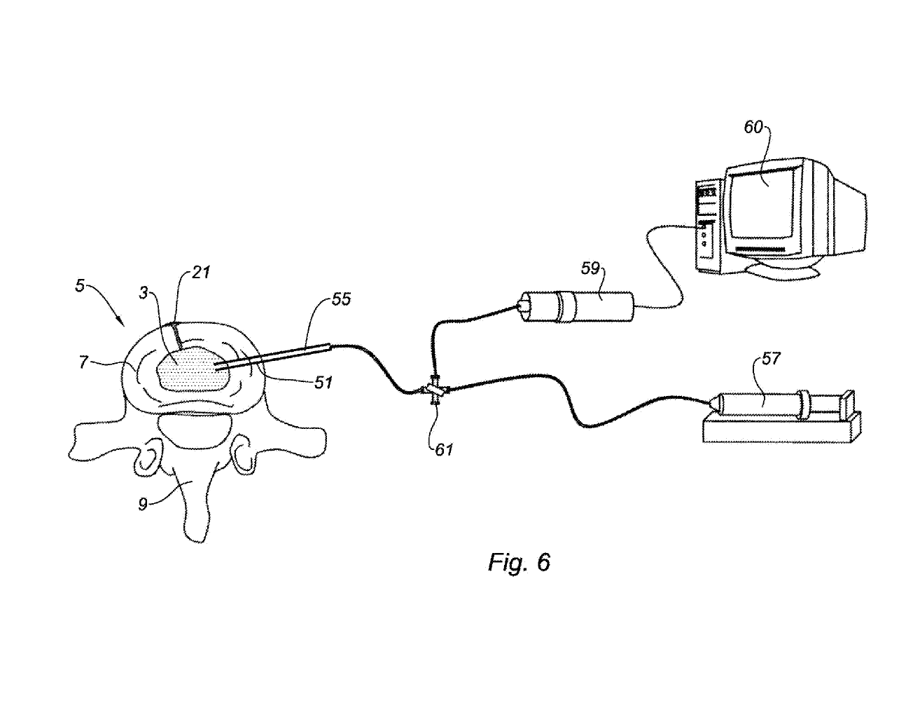

Annular passageways 51 are also made with the scalpel that are intended to receive tubes or sensors 53 (see FIG. 6).

The sample thus prepared is fixed and in one of the annular passageways 51 made in the annulus fibrosis 7 a flexible tube 55 is inserted to about 1 cm laterally on each side of the incision and to about 0.75 cm depth-wise. The tube 55 is connected to a pressurised syringe 57 allowing the injection via the tube 55 of a saline solution containing methylene blue into the nucleus. The syringe 55 is adjusted to about 3 ml·min⁻¹.

The saline solution is prepared by diluting about 2.16 g of sodium chloride in about 250 ml and adding methylene blue for staining. The staining of the saline solution intended to be injected into the central portion of the spinal disc allows for visual identification when the tissue defect leaks liquid.

During the injection the intra-disc pressure is measured with a pressure sensor 59 which can be connected to a data processor unit 60 of computer type. So as only to use one annular passageway 51, it is possible to use a bypass device 61 of Y-type to choose between injection and pressure measurement.

A healthy disc 5, namely having no lesion, has an annulus fibrosis 7 capable of deforming elastically to contain the saline solution. On the other hand, a disc which has suffered disc herniation and has been repaired has weakening at the point of the incision, which translates as the leaking of saline solution through the repaired incision if the repair has insufficient efficacy. For the comparative example, the pressure is measured to detect the maximum pressure. This maximum pressure corresponds to the maximum quantity of injected saline solution which can be retained by the annulus fibrosis. Over and above the maximum pressure, the saline solution leaks outside the said annulus. Therefore the measurement of the maximum pressure allows detection of the robustness and fluid-tightness of the repair of the incision.

The maximum intra-disc pressure that a healthy spinal disc can withstand is generally more than 32 bars, which therefore corresponds to the maximum pressure used for this example. Typically, the injected volume of the saline solution before being expelled by a healthy annulus is substantially equal to 5 ml.

Results of Pressure Measurement

For each type of disc whether or not repaired, about fifteen tests were performed. The value given below corresponds to the mean of the results obtained for the said fifteen tests.

The measured maximum pressure corresponds to the pressure of the saline solution beyond which the said solution leaks outside the annulus fibrosis of the spinal disc.

The spinal disc comprising the non-repaired incision withstands a maximum pressure of about 4.4 bars to within 1.5 bars.

The spinal disc comprising the incision repaired according to the Gold Standard method withstands a maximum pressure of about 10 bars to within 5 bars.

The spinal disc comprising the incision repaired with the assembly of the invention withstands a maximum pressure of about 25 bars to within 2 bars.

As indicated in the foregoing, a healthy spinal disc withstands a pressure of more than 32 bars, in particular substantially equal to 34.5 bars.

Therefore, the spinal disc repaired with the assembly of the invention has a weld which withstands a pressure close to the pressure that can be withstood by a healthy spinal disc, namely having a non-damaged annulus. As a result, the weld formed by means of the assembly of the invention is particularly efficient.

This invention is not limited to the repair of a herniated disc but can also be used for the repair of cartilage or another fibrous connective tissue.

1. An aqueous composition for use in the repair treatment of fibrous connective tissue by laser, comprising:
gold nanoparticles at a concentration of more than 1.5·10⁹ particles per ml and strictly less than 1·10¹⁰ particles per ml; and
albumin and/or collagen at a weight concentration of between 30% and 45%.

2. The composition according to claim 1 wherein the albumin and/or collagen is of bovine or human origin.

3. The composition according to claim 1 wherein the gold nanoparticles are of spherical, triangular or rod shape.

4. The composition according to claim 1 wherein the largest dimension of the gold particles is between 3 nm and 250 nm.

5. The composition according to claim 1 wherein the fibrous connective tissue is a cartilage, a ligament or a tendon.

6. An assembly comprising:
an aqueous composition according to claim 1;
a laser emitting at a wavelength of between 0.78 μm and 1.4 μm.

7. The assembly according to claim 6 wherein the laser emits at a wavelength of about 808 nm.

8. The assembly according to claim 6 wherein the laser is pulsed or continuous.

9. The assembly according to claim 6 wherein the laser is configured to have a beam with a diameter of between 3 mm² and 7 mm².

10. The assembly according to claim 9 wherein the laser is associated with a collimator and a fibre optical system.

11. The assembly according to claim 6 wherein the laser is configured to have an angle of incidence to a normal of an impact surface of the fibrous tissue of between 45° and 60°.

12. The composition according to claim 2, wherein the gold nanoparticles are of spherical, triangular or rod shape.

13. The composition according to claim 2 wherein the largest dimension of the gold particles is between 3 nm and 250 nm.

14. The composition according to claim 3 wherein the largest dimension of the gold particles is between 3 nm and 250 nm.

15. The composition according to claim 2 wherein the fibrous connective tissue is a cartilage, a ligament or a tendon.

16. The composition according to claim 3 wherein the fibrous connective tissue is a cartilage, a ligament or a tendon.

17. The composition according to claim 4 wherein the fibrous connective tissue is a cartilage, a ligament or a tendon.
